(19) United States
(12) Patent Application Publication
(10) Pub. No.: US 2023/0091812 A1
(43) Pub. Date: Mar. 23, 2023

(54) WHITE, BACTERIA-RESISTANT, BIOCOMPATIBLE, ADHERENT COATING FOR IMPLANTS, SCREWS AND PLATES INTEGRATED IN HARD AND SOFT TISSUE AND PRODUCTION METHOD

(71) Applicant: Ralf Masur, Bad Wörishofen (DE)

(72) Inventor: Detlev Repenning, Reinbek (DE)

(21) Appl. No.: 17/795,303

(22) PCT Filed: Jan. 25, 2021
(86) PCT No.: PCT/EP2021/051602

§ 371 Date: Jul. 26, 2022

(30) Foreign Application Priority Data
Jan. 27, 2020 (DE) 10 2020 101 882.7

(51) Int. Cl. A61C 8/00; C23C 14/02; C23C 14/00; C23C 14/08; C23C 14/16

(52) U.S. Cl. CPC A61C 8/0015; C23C 14/025; C23C 14/0084; C23C 14/083; C23C 14/16; C23C 14/027

(57) ABSTRACT

The invention relates to a white, bacteria-resistant, biocompatible, adherent coating for an element which can be integrated in hard and soft tissue, in particular an implant, a screw or a plate, having a structure made from metalliferous gradient layers having varying oxygen content, wherein the band gap of the outer-most gradient layer is greater than 3.1 eV, wherein the outer-most gradient layer is crystalline and wherein the gradient layers comprise tantalum and/or niobium and/or zirconium and/or titanium.

The present invention relates to a white, bacteria-resistant, biocompatible, adherent coating for implants, screws and plates which can be integrated in hard and soft tissue, an associated production method as well as to an implant having such a coating.

Coatings in particular fulfilling requirements with respect to bacterial resistances, relative to biocompatibility conditions, and in relation to chemical resistances are described in multiplicity. The following publications can be mentioned by way of example: R. Thull, Elektrochemische Prufung von (Ti,Nb)ON beschichteten Legierungen, [electrochemical testing of alloys coated with (Ti,Nb)ON], Biomedizinische Technik, Vol. 36(9), pages 214 and following, 1991; W. W. Plitz, Reib-und Verschleißuntersuchungen von (Ti,Nb)ON Schichten [friction and wear examinations of(Ti,Nb)ON layers], Labor Biomechanik, Munich, 1994; R. Thull, K. Taubner, E. J. Kahle, biologische und tierexperimentelle Untersuchungen (Ti,Zr)O₂ und (Ti,Nb)ON [biological and animal experiment examinations (Ti,Zr)O₂ and (Ti,Nb)ON], Biomedizinische Technik, Vol. 37, 7-8, 1992; R. Thull, K. Taubner, E. J. Kahle, Modell zur immunologischen Prufung von Biomaterialien [Model for immunologically testing biomaterials], Biomedizinische Technik, Vol. 37, 162-169, 1992; D. Repenning, Materialempfindlichkeit bei Titanimplantaten [material-sensitivity in case of titanium implants]—part 1, Zahnheilkunde Management ZMK, 2010; M. Stelzel et al., Verhalten verschiedener Titanoberflachen bei oraler Exposition [behavior of different titanium surfaces in case of oral exposition]—an in vivo study, Philips University, Marburg, Dept. Parodontology 2003; R. Repenning, Oberflächenbeschichtung auf Implantaten, ossäre Integration [surface coating on implants, osseous integration]53-61, Springer Verlag; Betz, Reuther, 5-jährige klinische Studie enossaler Implantate unter besonderer Berücksichtigung des periimplantären Gewebes [5 years lasting study of enossal implants while particularly considering the periimplant tissue], Deutsche Zeitschrift für Mund- und Kieferchirurgie [German Trade Journal for oral and maxillofacial surgery], 1995.

All of these layers have the disadvantage that they do not fulfil the aesthetic criterion of a white surface. In the present context, under “white”, a colour domain coloured between slightly grey and slightly pink up to purely white, which is perceived in the respective organic environment as white, is to be understood in this case.

Especially for coated dental implants of titanium there is the desire to provide aesthetically white surfaces. The same applies both for the enossal part of the implant and the abutment. At the same time, the surfaces are subject to the requirements of biocompatibility (ELISA, Thull) mentioned above, as well as to the requirements of an effective corrosion protection against ions exiting the substrate material and a bacteria resistance, in particular against pathogenic, anaerobic bacteria. Not least, a topographically optimally adjusted surface needs to ensure a safe osseointegration.

The hitherto available coatings have colour appearances from gold tones (nitrides) via dark blue (ceramic titanium zirconium oxide layers) up to black (DLC=diamond like carbon). Known white-grey coatings are represented by hydroxyapatite coatings (HA coatings), which serve for better osteointegration. These HA coatings, however, are temporally resorbed in the tissue. Moreover, they are only applied onto the enossal part of an implant and not onto the transgingival implant part.

Titanium dioxide coatings which are white or similar to white produced by electrolytical methods have also become to be known. However, titanium dioxides do not fulfil the requirements for bacteria resistance mentioned above (see M. Stelzel et al., Verhalten verschiedener Titanoberflachen bei oraler Exposition [behavior of different titanium surfaces in case of oral exposition]—an in vivo study, Philips Universität, Marburg, dept. Parodontologie 2003), and only to a low extent those for biocompatibility (see R. Thull, K. Taubner, E. J. Kahle, biologische und tierexperimentelle Untersuchungen (Ti,Zr)O₂ und (Ti,Nb)ON [biological and animal experiment examinations (Ti,Zr)O₂ and (Ti,Nb)ON], Biomedizinische Technik, Vol. 37, 7-8, 1992). Further known approaches for coating implant bodies are based on sol-gel processes and spraying processes. Plasma spraying (APS), cold spraying (CGS), high velocity oxygen fuel spraying (HVOF), arc spraying (AS), powder flame spraying (PFS), and last but not the least wire flame spraying are part of these processes. All of these layers suffer from the disadvantage that they cannot be applied in a sufficiently adhesive manner for medical application or/and cannot be applied onto the filigree implant bodies with sufficient precision.

White dental implants are currently provided from full ceramics of zirconium dioxide. For stabilizing against fracture, the ceramics are doped with yttrium oxide. Ceramic implants especially have the disadvantage that their surfaces topographically cannot be adjusted optimally for a safe osseointegration, and that they rise a particular and increased challenge to surgery and long-term stability (or premature losses in the first year) due to the risk of fracture. Moreover, the compositions of the ceramics cannot be freely variated for material-technically systematic reasons, and thus do not enable the material to be adequately adapted to the biological environment.

Against the background of the above explanations, a task of the invention is to overcome the mentioned disadvantaged of the state of the art and to proposes a coating for implants both fulfilling the aesthetic criterion with respect to the surface colour and having good biocompatibility, long-term stability and bacteria resistance.

This task is solved by a coating having the features of claim 1. The task is further solved by a methods of producing a coating having the features of claim 12, and an implant having the features of claim 21.

Advantageous further developments will result from the subclaims.

An essential ideal of the invention is to structure the coating in several layers from gradient layers, wherein the oxygen content varies among the gradient layers. Preferably, the lowermost gradient layer applied onto the element is formed as a metallic adhesive bonding layer. Within the scope of the present invention, a metallic adhesive bonding layer is understood as a gradient layer of a metal and being substantially free from oxygen. This enables an optimum adhesion on the element to be achieved. The gradient layers formed on the metallic adhesive bonding layer preferably have an oxygen content increasing from the lowermost gradient layer applied onto the element to the outermost gradient layer to the full stoichiometry such that the outermost gradient layer is a metal oxide layer having full stoichiometry.

By means of gradually increasing the oxygen content via the gradient layers, the adhesive bonding between the gradient layers is improved. Both the metallic adhesive bonding layer and the gradient layers having an oxygen content below the full stoichiometry of the outermost metal oxide gradient layer have a coloration caused by defects in the crystal structure within the gradient layers having an oxygen content, where electronic intermediate states arise resulting in a characteristic absorption and thus to a coloration of the material. The outermost gradient layer having full stoichiometry is crystalline and substantially has no defects in the structure causing coloration. Thereby, a coating is produced, on the one hand, which due to the gradually varying oxygen content from the metallic adhesive bonding layer to the outermost metal oxide gradient layer is characterized by excellent adhesive bonding properties and simultaneously has a white coloration due to the presence of the metal oxide outermost gradient layer.

The band gap of the outermost gradient layer is greater than 3.1 eV, so that the outermost layer does not absorb electromagnetic waves in the visible range and thus appears to be white.

Furthermore, materials, in particular ceramic systems, having band gaps of more than 3.1 eV, represent materials having a high electric resistance (R>>1000 Ωcm²) and contribute in avoiding the reaction between the biological tissue and an implant formed of the material by ion exchange.

The bacteria resistance, biocompatibility and adhesive strength achieved according to the invention due to the coating is advantageous for all of the elements used in conjunction with an implantation. Therefore, the coating according to the invention is applicable for the entirety of implant components. It should be pointed out at this point in a clarifying manner that the coating according to the invention is suitable for the entirety of elements and their surfaces, which can be integrated into hard and soft tissues or can be connected to such elements, for example, to all of the surfaces of implants, abutments and connecting elements such as screws, including their entire outer surfaces as well as inner surfaces including thread portions.

Preferably, at least one of the gradient layers having an oxygen content, in particular at least the outermost metal oxide gradient layer has grain sizes of 5 nm or greater. The nanocrystalline structure having grain sizes in the submicron range contributes to an increased fracture toughness of the layers. The determination of the grain sizes is performed by means of X-ray diffractometry.

Preferably, the gradient layers have tantalum and/or niobium and/or zirconium and/or titanium. These metals are characterized by high biochemical stability.

According to the method according to the invention, the application of the coating preferably is performed by means of a PVD (physical vapour deposition) process. PVD processes are typically used for the coating of medical implants and instruments. Their advantage is the high variability to adjust chemical compounds, be it purely metallic, oxidic, nitride, carbide or more complex compositions.

According to the invention, oxidic, at least nanocrystalline layers are produced by means of PVD processes, wherein the band gap E_{g} of at least the outermost gradient layer is greater than 3.1 eV. The adhesion of the layers is achieved by means of gradient layers in that firstly a metallic adhesion bonding layer is applied onto the element, and in the following, the oxygen content of the layers is adjusted to the full stoichiometry, preferably within less than 500 nm. It is essential that the gradient layers are adjusted to be free from defects insofar that no electron states arise resulting either in reducing the band gap or serving as absorption centres for light of longer wavelengths.

A particular disadvantage of the PVD processes, namely that the deposition conditions are outside the thermodynamic balance, is eliminated in that the coating is made at an increased temperature of above T=300° C. and/or that the layers are cured subsequently under an oxygen atmosphere. The additionally particular claim with respect to the bacteria resistance is preferably achieved via the multiphase adjustment of the layers. Multiphase layers vary at the surface the pzzp (point of zero zeta potential) with the effect of bacterial repulsion.

In the following, the invention will be described also with respect to further features and advantages on the basis of exemplary embodiments, which are explained in more detail on the basis of the FIGURE.

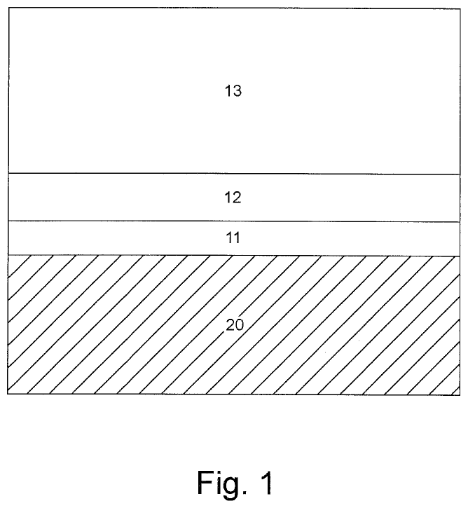

In this case, FIG. 1 shows a schematic view of an element having a coating according to the invention.

FIG. 1 shows an element 20 formed by an implant such as a screw or plate that can be integrated into hard and soft tissue. A coating according to the invention made of several gradient layers 11, 12, 13 is applied onto the element. The lowermost gradient layer 11 formed on the element 20 is a metallic adhesion bonding layer. The outermost gradient layer 13 is a white layer containing a metal oxide having full stoichiometry. Between the gradient layers 11 and 13, one or more gradient layer/s 12 are formed, the oxygen content of which increases from the lowermost gradient layer 1 formed on the element 20 up to the outermost gradient layer 13 with the full stoichiometry.

In the simplest case, a white layer of zirconium dioxide is adjusted. Zirconium dioxide is used in the implantology as a full ceramic material. Up to now, however, it has not worked to apply zirconium dioxide onto titanium implant bodies as a tightly adherent white layer. In the embodiment according to the invention, firstly a metallic layer is deposited in a layer thickness of 20 nm as the lowermost gradient layer 11 of zirconium on the element 20, the surface of which preferably is roughened and formed of titanium in a preferred embodiment. In the following, the oxygen is successively supplied by means of a PVD typical reactive process, and the layer is finally formed in the full stoichiometry present in the outermost gradient layer 13, via the gradient layers 12. The entire layer thickness of the coating is preferably adjusted to 5 micrometres. The process control may be made such that the deposition takes place at an increased temperature so that the otherwise PVD typical (x-ray amorphous) layer is not generated but an at least nanocrystalline layer.

In a second exemplary embodiment, a mixed phase having 20 mol % of Nb₂O₅ and 80 mol % of Ta₂O₅ is adjusted for the outermost gradient layer 13, and the full stoichiometry is established starting from the lowermost, metallic gradient layer 11 via the intermediate gradient layers 12. The layers are characterized by a particularly high biochemical stability and a negative surface potential. The negative surface potential causes a stable adsorption of calcium ions and a therewith associated safe osteointegration.

In the further example, a layer having the stoichiometry of ZrTi₂O₆ is used as the outermost gradient layer 13. (Ti,Zr)O₂₋ₓ layers are highly biocompatible and blue-black due to their high defect structure, their x-ray amorphous morphology and their non-exact composition adjustment. The coating according to the invention has an outer gradient layer 13 having a band gap E_{g} of 3.1 eV or more, and is adjusted to be exact in stoichiometry. This gradient layer 13 is nanocrystalline. Due to its high negative free enthalpy of formation, the outermost gradient layer 13 in addition has a significantly improved biochemical stability. Its point of zero potential is at pH 6-7.

1-21. (canceled)

22. An implant formed by a dental implant having an enossal part and an abutment,
characterized in that
a white, bacteria-resistant, biocompatible, adherent coating is applied onto both an enossal part and the abutment of the dental implant, wherein the coating has a structure made from metalliferous gradient layers (11, 12, 13) having a varying oxygen content, wherein the band gap (E_{g}) of the outermost gradient layer (13) is greater than 3.1 eV, wherein the outermost gradient layer is crystalline, and wherein the gradient layers (11, 12, 13) comprise tantalum and/or niobium and/or zirconium and/or titanium.

23. The implant according to claim 22, wherein the lowermost gradient layer (11) applied onto the implant (20) is a metallic adhesive bonding layer, the outermost gradient layer (13) is a metal oxide layer having full stoichiometry, and wherein the intermediate gradient layers (12) have an oxygen content increasing from the lowermost gradient layer (11) applied onto the implant to the outermost gradient layer (13) to the full stoichiometry.

24. The implant according to claim 22, wherein at least one of the gradient layers (12, 13) having an oxygen content, preferably at least the outermost gradient layer (13), has grain sizes of 5 nm or greater.

25. The implant according to claim 22, wherein the gradient layers (11, 12, 13) further have aluminium and/or tin.

26. The implant according to claim 22, wherein the concentration of the metals in the gradient layers (12, 13) is adjusted by at least binary oxides such that the gradient layers (12, 13) having at least binary oxides have a band gap (E_{g}) of greater than 3.1 eV.

27. The implant according to claim 22, wherein one or more gradient layer/s (11, 12, 13) contain/s carbon and/or nitrogen and/or boron and/or fluor.

28. The implant according to claim 22, wherein the lowermost gradient layer (11) applied onto the implant has a thickness of 50 nm or less.

29. The implant according to claim 22, wherein the entire thickness of the gradient layers (12) having a reduced oxygen stoichiometry amounts to 500 nm or less, preferably 200 nm or less, further preferably 100 nm or less, further preferably 60 nm or less.

30. The implant according to claim 22, wherein the thickness of the outermost gradient layer (13) amounts to 10 μm or less.

31. The implant according to claim 22, wherein the entire thickness of the coating amounts to between 3 μm and 7 μm, preferably between 4 μm and 6 μm, further preferably between 4.5 μm and 5.5 μm.

32. A method for producing a white, bacteria-resistant, biocompatible, adherent coating on an implant according to claim 22, comprising the following steps:
applying a metallic adhesion bonding layer as a first gradient layer (11) onto the surface of the implant (20) by means of PVD (physical vapor deposition),
applying gradient layers (12, 13) comprising tantalum and/or niobium and/or zirconium and/or titanium, as well as oxygen, onto the metallic adhesion bonding layer (11) having an increasing oxygen content by increasing the oxygen content during the application of the gradient layers (12, 13) until the full stoichiometry of the outermost gradient layer (13) is reached,
wherein the band gap (E_{g}) of the outer gradient layer (13) is greater than 3.1 eV.

33. The method according to claim 32, wherein the application of the gradient layers (11, 12, 13) is performed at a temperature of 300° C. or higher.

34. The method according to claim 32, wherein the gradient layers (11, 12, 13) are cured under an oxygen atmosphere.

35. The method according to claim 32, wherein the gradient layers (11, 12, 13) are formed such that they have grain sizes of 5 nm or greater.

36. The method according to claim 32, wherein the gradient layers (11, 12, 13) comprise tantalum and/or niobium and/or zirconium and/or titanium, as well as oxygen.

37. The method according to claim 32, wherein the gradient layers (11, 12, 13) further comprise aluminium and/or tin.

38. The method according to claim 32, wherein the application of the gradient layers (11, 12, 13) is performed such that the lowermost gradient layer (11) applied onto the implant (20) is a metallic adhesive bonding layer, the outermost gradient layer (13) is a metal oxide layer having full stoichiometry, and wherein the intermediate gradient layers (12) have an oxygen content increasing from the lowermost gradient layer (11) applied onto the implant to the outermost gradient layer (13) to the full stoichiometry.

39. The method according to claim 32, wherein the concentration of the metals in the gradient layers (12, 13) is adjusted by at least binary oxides such that the gradient layers (12, 13) having at least binary oxides have a band gap (E_{g}) of greater than 3.1 eV.

40. The method according to claim 32, wherein one or more gradient layer/s (11, 12, 13) contain/s carbon and/or nitrogen and/or boron and/or fluor.
